(19) United States
(12) Patent Application Publication
(10) Pub. No.: US 2004/0022749 A1
(43) Pub. Date: Feb. 5, 2004

(54) Nail enamel composition containing a primary film forming polymer, a citrate ester, and an aldehyde condensation product

(76) Inventor: Alain Malnou, Routot (FR)

Correspondence Address: Mathews, Collins, Shepherd & McKay, P.A., 100 Thanet Circle, Suite 306, Princeton, NJ, 08540, US

(21) Appl. No.: 10/387,853

(22) Filed: Mar. 13, 2003

(30) Foreign Application Priority Data
Jul. 23, 2002 (FR) 02.09315

(51) Int. Cl. A61K 7/04

(52) U.S. Cl. 424/61

(57) ABSTRACT

A nail enamel composition containing a primary film forming polymer, a citrate ester and an aldehyde condensation product.

Priority is claimed under 35 USC §119 from National Application No.02.09315 filed in France on Jul. 23, 2002.

### FIELD OF THE INVENTION

The present invention relates in general to nail enamel compositions formulated without formaldehyde, aromatic solvents, and/or phthalic components.

### BACKGROUND

Nail enamels conventionally consist of a main film-forming polymer, which is generally made of a nitrocellulosic polymer, plasticizers of this main polymer, secondary polymers, and solvents. These compositions also frequently utilize pigments, suspending agents, and additives intended as UV quenchers or vitamins.

The objectives of these compositions are to obtain films, that when laid down on a nail, will dry quickly, be glossy, and have resistance to wearing and chipping. Quick drying is obtained by the use of cellulosic polymers and gloss by plasticizers and secondary resins. The compromises of hardness vs. flexibility and wearing vs. chipping are optimized by the choice and quantity of the plasticizers and secondary resins used with the cellulosic polymers.

Phthalic ester plasticizers, when utilized with nitrocellulosic polymers exhibit good flexibility and mechanical properties. But, because of toxicological problems, these plasticizers must be superseded. Citrate esters are good candidates for this. (EP-A-0 613 676)

One secondary resin, well known in the nail varnish industry for its gloss quality, adhesion, and elasticity, is the condensate of tosylamide with formaldehyde. However, free formaldehyde is found in varnishes made with this condensate. Formaldehyde is an allergenic product, so a substitute must be found for this particular resin.

The objective of this work is to replace these products, and to find plasticizers and resins with similar mechanical properties or better.

For toxicological reasons, the use of aromatic solvents is suspect in nail enamels.

### REFERENCES

| | | |
|---|---|---|
| 5,662,891 | September 1997 | Martin |
| 5,807,504 | September 1998 | Junino |
| 5,882,636 | March 1999 | Mui et al. |
| 5,977,217 | November 1999 | Socci et al. |
| 6,110,447 | August 2000 | Ramin et al. |
| 6,126,952 | October 2000 | Socci et al. |
| 6,333,025 | December 2001 | Ramin et al. |

Foreign Patent Documents

| | | |
|---|---|---|
| 154679 | September 1985 | EP |
| 0613676 | September 1994 | EP |

### SUMMARY OF THE INVENTION

Described is a formulation for nail varnish composed of:

a) at least a film-forming cellulosic polymer,

b) at least a citric acid ester, and

c) at least a condensate resin made with one or several aldehydes excepting formaldehyde.

This invention contains no formaldehyde, no aromatic solvents, and no phthalate condensation products, facts seen as improvements upon previously existing blends.

### DESCRIPTION OF THE INVENTION

One of the objectives of this invention is to create a composition without phthalate products, aromatic solvents, and/or free formaldehyde, with better characteristics than a composition containing citrate only (EP-A-0 613 676), and desirable properties at least equal to those of phthalate products.

During experimental trials, in obtaining the best compromises of hardness, flexibility, drying, adhesion, and durability on nails, the inventor discovered that blending citrate esters with a non-formaldehyde aldehyde condensation product gave excellent adhesion, durability, and gloss results.

Then, according to the invention, the cosmetic composition for a nail varnish free of phthalic products, formaldehyde, and aromatic solvents, consists of:

| | |
|---|---|
| Component A): | at least a cellulosic film-forming polymer, |
| Component B): | at least a citric ester product as a plasticizer, and |
| Component C): | at least a condensate resin using one or several aldehydes |
| | other than formaldehyde. |

This composition, comprised of at least 3 ingredients and when used as nail varnish, gives a glossy, homogeneous film that dries quickly and has better mechanical properties and durability on fingernails than a formulation without these three components. In particular, these nail enamels have equal or better cosmetic properties than those with phthalic components or formaldehyde condensation products.

The cellulosic film-forming derivative (Component A) chosen is nitrocellulose (in particular, nitrocellulose type RS [not soluble in alcohols but soluble in esters and ketones] with differing viscosities), the acetopropionate of cellulose and cellulose ethers as ethyl or methyl cellulose, or a mixture of these components.

Among the citric acid esters used are: tri-ethyl or tri-butyl citrate, tri-(2-ethyl hexyl) citrate, acetyl tri-ethyl citrate, acetyl tri-butyl citrate, or acetyl tri-(2-ethyl hexyl) citrate, or some mixture thereof.

Preferred usage is nitrocellulose with acetyl tri-butyl citrate.

The condensate resins produced with one or several aldehydes (excepting formaldehyde) used to advantage are:

Ether resins, the result of a condensation between a polyvinylic alcohol and an aldehyde, preferably one with at least 3 carbons, for example propionaldehyde, butanal, heptanal and/or an aromatic aldehyde like benzaldehyde

An aliphatic aldehyde poly condensate composed of at least 2 carbons acetaldehyde, propionaldehyde, . . . ) and/or an aromatic aldehyde such as benzaldehyde, with an ammoniac derivative such as urea.

Some mixture of these.

Some of these resins are industrially available and are trademarked, for example, Mowital (Hoechst) or Laropal (BASF).

In preparation, the weight of the citric acid ester (Component B) is between 1 to 100% of the weight of the cellulosic polymer, preferably between 40 and 80% by weight, and the weight of the aldehyde condensate (Component C) is between 1 and 120% of the cellulosic polymer, preferably between 10 and 80% by weight.

To these three main components of the invention, we can add other film-forming resins such as: acrylic copolymers, polyesters modified or not by fatty acids, epoxy, acrylics, vinyls or amids, polyurethanes modified or not by acrylics, polyesters, polyethers or polyureas, and natural resins such as Arabic, guars, or dammars gums.

We can also change the elasticity with other plasticizers: sucrose acetate, isobutyrate, diol benzoates, tri-cresyl phosphate, or tri-glyceryl acetate.

To prepare solutions with these polymers, the preferred uses are aliphatic acetates as ethyl, butyl, iso-butyl, propyl, or amyl acetate. We can also add ketones (methyl ethyl ketone, methyl iso-butyl ketone, di-acetone alcohol), alcohols (ethanol or iso-propanol), or aliphatic mineral spirits. Solvents with aromatic components are not used.

To obtain the desired colors, we can mill in pigments and dyes used for cosmetic products: inorganic pigments, metallic particles, organic pigments, lakes, pearlescent pigments, natural glitters, synthetic multi-layered particles, and extenders, both organic and inorganic.

To create good theological characteristics, we can mill into the resin solution modified hectorite or bentonite, pyrogenic silica, polyurea/urethane particles, or wax as hydrogenated castor oil.

The ingredients for nail varnishes, namely, the components of the invention, are made with classical techniques known in the field.

The present invention also describes the use of these mixtures as nail varnishes, applied as a single product with one or several layers, as an undercoating on nails, as a top coat, or as an intermediate coat.

### EXAMPLES

The present invention is described further in the following examples, which are intended for the purposes of illustration and are not limiting as to other possible uses. The present compositions are prepared according to classical directions for use. The formulas are expressed as percentage by weight.

### Example 1

### STD 1

This comparative formula is a classical formula using phthalic plasticizer, toluol, and formaldehyde.

TABLE I

| | % |
|---|---|
| Nitrocellulose (RS ¼ second) | 12.6 |
| Isopropanol | 5.4 |
| Di-butyl Phthalate | 7.2 |
| Tosylamide/Formaldehyde Condensate | 5.6 |
| Stearalkonium Hectorite | 1.2 |
| Ethyl Acetate | 30.5 |
| Butyl Acetate | 25.0 |
| Toluol | 10.0 |
| Red 7 Lake | 0.2 |
| Red 34 Lake | 0.3 |
| Mica Titane Pigment | 2.0 |
| Total: | 100.0 |

### Example 2

### STD 2

This is a comparative formula with citrate and without aldehyde condensate.

TABLE II

| | % |
|---|---|
| Nitrocellulose (RS ¼ second) | 12.6 |
| Isopropanol | 5.4 |
| Acetyl tri-butyl Citrate | 7.2 |
| Polyester Resin (without phthalic products) | 5.6 |
| Stearalkonium Hectorite | 1.2 |
| Ethyl Acetate | 38.5 |
| Butyl Acetate | 27.0 |
| Red 7 Lake | 0.2 |
| Red 34 Lake | 0.3 |
| Mica Titane Pigment | 2.0 |
| Total: | 100.0 |

### Example 3

### Formula with Citrate and Polyvinyl Butyral

TABLE III

| | % |
|---|---|
| Nitrocellulose (RS ¼ second) | 12.6 |
| Isopropanol | 5.4 |
| Acetyl tri-butyl Citrate | 7.2 |
| Mowital ® B30 (Hoechst Polyvinyl Butyral) | 1.0 |
| Polyester Resin (without phthalic products) | 4.6 |
| Stearalkonium Hectorite | 1.2 |
| Ethyl Acetate | 38.5 |
| Butyl Acetate | 27.0 |
| Red 7 Lake | 0.2 |
| Red 34 Lake | 0.3 |
| Mica Titane Pigment | 2.0 |
| Total: | 100.0 |

### Example 4

### Formula with Citrate and Urea/Aldehyde Condensate

TABLE IV

| | % |
|---|---|
| Nitrocellulose (RS ¼ second) | 12.6 |
| Isopropanol | 5.4 |
| Acetyl tri-butyl Citrate | 7.2 |
| Laropal ® A101 (BASF Urea Aldehyde) | 5.6 |
| Stearalkonium Hectorite | 1.2 |
| Ethyl Acetate | 38.5 |
| Butyl Acetate | 27.0 |
| Red 7 Lake | 0.2 |
| Red 34 Lake | 0.3 |
| Mica Titane Pigment | 2.0 |
| Total: | 100.0 |

The physical properties of these films are very difficult to measure on nails, so trials are carried out on other surfaces, such as glass or aluminium, to compare formulas. Films are laid down with a Conway applicator to have a liquid thickness of 100 microns. The drying times are recorded on a thermo-stable plate at 35° C. The drying time is given by a print leave from a small sphere running on a film applied to a Leneta card. These physical tests are executed after an overnight drying at 20° C. or 2 hours at 60° C.

The film thickness is measured according to ISO 2360 on aluminum. The film gloss is measured with a gloss meter (at a 60° angle) according to ISO 2813. The hardness is measured on glass with a Persoz pendulum apparatus according to ISO 1522. Adhesion is measured on glass with a 1 mm square cutting followed by pulling up with an adhesive tape according to ISO 2409.

Finally, a durability test is organized on a testing group to evaluate gloss, wear and chipping.

For these examples the results are:

TABLE V

| | Std 1 | Std 2 | 1 | 2 |
|---|---|---|---|---|
| Drying (min) | 4 | 3.6 | 3.8 | 3.8 |
| Hardness (sec) | 260 | 280 | 270 | 280 |
| Adhesion (0 to 5) | 0/1 | 1 | 0/1 | 0/1 |
| Durability (days) | 2.9 | 2.3 | 2.8 | 3.1 |

All these compositions demonstrated good adhesions in the range of 0 to 1 (0=Very Good, 5=Poor).

These results show that these new compositions, 1 and 2, have properties at least equivalent to those made with phthalic and formaldehyde condensate products. In particular, invention compositions 1 and 2 have good durability on nails, better than that of Std 2 (which is comprised of citrate only) and approximately equal to that of Std 1 (which is comprised of phthalates and formaldehyde). Moreover, the composition with citrate and urea/aldehyde condensate has better gloss.

What is claimed is:

1. A cosmetic formulation for nails without formaldehyde, aromatic solvents, and/or a phthalate product, the formulations contains:
a) at least a film-forming cellulosic polymer,
b) at least a citric acid ester, and
c) at least a condensate resin made with one or several aldehydes except formaldehyde.

2. A cosmetic formulation according to claim 1, wherein said cellulosic polymer is chosen from cellulose nitrates, cellulose aceto-butyrates, cellulose aceto-propionates, cellulose ethers and/or a mixture of these.

3. A cosmetic formulation according to claim 1, wherein said citric acid ester is chosen from tri-ethyl citrate, tri-butyl citrate, tri-(2-ethyl hexyl) citrate, acetyl tri-ethyl citrate, acetyl tri-butyl citrate, or acetyl tri-(2-ethyl hexyl) citrate.

4. A formulation, according to claim 1, wherein the cellulosic polymer is a cellulose nitrate and wherein the citric ester is acetyl tri-butyl citrate.

5. A formulation, according to claim 1, wherein the condensate resin (from claim 1.c) is a resin resulting from the condensation of polyvinyl alcohol with an aliphatic aldehyde, branched or not, containing at least 3 carbon atoms; and/or an aromatic aldehyde.

6. A formulation, according claim 1, wherein the condensate resin (from claim 1.c) is a poly condensate resin from aliphatic aldehydes containing at least 2 carbon atoms and/or aromatic aldehydes, with an ammoniac derivative.

7. A formulation according to claim 5 or 6, wherein the aldehyde is chosen from propionaldehyde, butyraldehyde, heptanal, hexanal or benzaldehyde.

8. A formulation according to claim 6, wherein the ammoniac derivative is urea.

9. A formulation according to claim 1, wherein the weight of the citrate ester is between 1 to 100% of the weight of the cellulosic polymer, preferably between 40 and 80%.

10. A composition according to claim 1, wherein the weight of the condensate resin (from claim 1.c) is between 1 and 120% of the cellulosic polymer, preferably between 10 and 40%.

11. A composition according to claim 1, wherein is comprised of at least a component chosen among dyes, inorganic or organic pigments, decorative glitters, inorganic or organic extenders, anti-settling agents, UV quenchers, anti-oxidizing agents, and/or additives such as vitamins, proteins or extracts from plants, animals and/or minerals.

12. Use of the composition according to claim 1 as a nail enamel, applied as a single product in one or several layers.

13. Use of the composition according to claims 1 as an undercoat, as a topcoat, or as a mid coat.
